(19) United States
(12) Patent Application Publication
(10) Pub. No.: US 2015/0072365 A1
(43) Pub. Date: Mar. 12, 2015

(54) MAGNETICALLY ALIGNING TEST STRIPS IN TEST METER

(71) Applicant: Cilag GmbH International, Zug (CH)

(72) Inventors: Brian GUTHRIE, Inverness (GB); Scott Sloss, Inverness (GB); Allan Macrae, Inverness (GB); Ruth Hunter, Inverness (GB)

(73) Assignee: Cilag GmbH International, Zug (CH)

(21) Appl. No.: 14/023,178

(22) Filed: Sep. 10, 2013

(51) Int. Cl. C12Q 1/54

(52) U.S. Cl. CPC C12Q 1/54
USPC 435/14; 435/287.1

(57) ABSTRACT

An analytical test meter includes a meter housing containing a test strip connector that includes at least two terminals. A processor is disposed within the meter housing, as well as a current generator that generates a magnetic field in association with one of the terminals for attracting a contact of an analytical test strip for alignment or retention therewith. Detection of the presence of an analytical test strip relative to an electrical contact can cause an increase in the intensity of the magnetic field.

### FIELD OF THE INVENTION

The present disclosure generally relates to systems used for determining analyte concentration of a test sample and more specifically to a test meter that is configured to apply a magnetic field for purposes of aligning or retaining analytical test strips.

### DESCRIPTION OF RELATED ART

Analyte detection in physiological fluids, e.g., blood or blood derived products, is of ever-increasing importance in today's society. Analyte detection assays find use in a variety of applications, including clinical laboratory testing, home testing, etc., in which the results of such testing play a prominent role in diagnosis and management in a variety of disease conditions. Analytes of interest include glucose for purposes of diabetes management, cholesterol, and the like. In response to this growing importance of analyte detection, a variety of analyte detection protocols and devices for both clinical and home use have been developed.

One method employed for analyte detection is that employing an electrochemical cell, typically provided in an analytical test strip. An aqueous liquid sample is placed into a sample-receiving chamber in the electrochemical cell, the cell typically employing two electrodes, e.g., a counter electrode and a working electrode. The analyte of interest is allowed to react with a redox reagent to form an oxidizable (or reducible) substance in an amount corresponding to that of the analyte concentration. The quantity of the oxidizable (or reducible) substance present is then estimated electrochemically and related to the amount of analyte present in the initial sample.

As noted, the electrochemical cell is typically present on a test strip which is configured to electrically connect the cell to an analyte measurement device. While current test strips are effective, the size of the test strips directly and relatedly impact the costs of manufacture. While it is desirable to provide test strips having a size that facilitates handling of the test strip, increases in size will tend to increase manufacturing costs where there is an increased amount of material used to form the strip. Moreover, increasing the size of the test strip tends to decrease the quantity of strips produced per batch, which also impacts manufacturing costs.

To that end, smaller analytical test strips have been produced. These test strips, however, can be difficult to handle given their smaller size especially in removing the test strip from a storage container and also in properly engaging or orienting the test strip with a test meter. While specific carriers can be developed for the handling of such test strips, this would add complexity and additional hardware to a test system.

Accordingly, there is a need in the field to develop an improved technique for the handling of the smaller test strips.

### BRIEF DESCRIPTION OF THE DRAWINGS

Various novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a perspective view of an exemplary integral analytical test strip;

FIG. 2 is a side elevational view, taken in section, of the integral analytical test strip of FIG. 1, together with related components;

FIG. 3 shows an exemplary electrochemical module;

FIG. 4 is a perspective view of an exemplary modular analytical test strip using the exemplary electrochemical module;

FIG. 5 is a perspective view and block diagram of a test meter and test strip according to an exemplary embodiment;

FIG. 6 is a perspective view showing an example of use of the test meter with a container of test strips;

FIG. 7 is a flow diagram depicting stages in a method for enabling a compact analytical test strip to be accurately inserted into a test meter according to various embodiments of the present invention; and

FIG. 8 is a perspective view of another exemplary embodiment of a test meter and test strip.

### DETAILED DESCRIPTION OF ILLUSTRATIVE EMBODIMENTS

The following description relates to exemplary embodiments for engaging and aligning an analytical test strip with a test meter. These exemplary embodiments are intended to provide an overall understanding of the principles of the structure, function, manufacture and use of the devices, systems, and methods disclosed herein. One or more examples of these embodiments are illustrated in the accompanying drawings, which are not necessarily to scale. Those skilled in the art will understand that the devices and methods specifically described herein and illustrated in the accompanying drawings are non-limiting exemplary embodiments and that the scope of the present disclosure is defined solely to the claims. The features illustrated or described in connection with one exemplary embodiment may be combined with the features of other embodiments. Such modifications and variations are intended to be included within the scope of the present disclosure.

In addition and throughout the course of discussion several terms, which can include “front”, “back”, “upper”, “lower”, “top”, “bottom”, “lateral”, and the like are used in order to provide a suitable frame of reference in regard to the accompanying drawings. These terms are not intended to limit scope, unless specifically indicated herein.

In addition, a person skilled in the art will further appreciate that the terms “about” and “approximately”, as used herein for any numerical value or ranges of numerical values, merely provide a suitable dimensional tolerance that allows the component or collection of components to function for its intended purpose.

Throughout this description, some embodiments are described in terms that would ordinarily be implemented as software programs. Those skilled in the art will readily recognize that the equivalent of such software can also be constructed in hardware (hard-wired or programmable), firmware, or micro-code. Given the systems and methods as described herein, software or firmware not specifically shown, suggested, or described herein that is useful for implementation of any embodiment is conventional and within the ordinary skill in such arts.

In general, test meters, such as hand-held test meters, configured to receive an analytical test strip for determining an analyte concentration of a fluid sample, include a meter housing, a strip port connector, a processor, and a field generator configured to provide a magnetic field that will draw a magnetic material on the analytical test strip towards at least one terminal of the strip port connector. Test meters according to embodiments of the present invention are beneficial in that they permit grasping and retaining test strips. This permits, e.g., using smaller test strips than an average human user can comfortably handle. Smaller test strips can be less expensive and, with magnetic grasping as described herein, more convenient than conventional test strips.

A problem solved by various embodiments is that users can have difficulty handling and manipulating small test strips, especially in locating or otherwise positioning same properly with a test meter to enable an analyte measurement to be reliably taken in a repeatable manner. Various embodiments also use magnetic fields to correctly align test strips so that, e.g., the working and counter electrodes are connected to the test meter with the correct polarity.

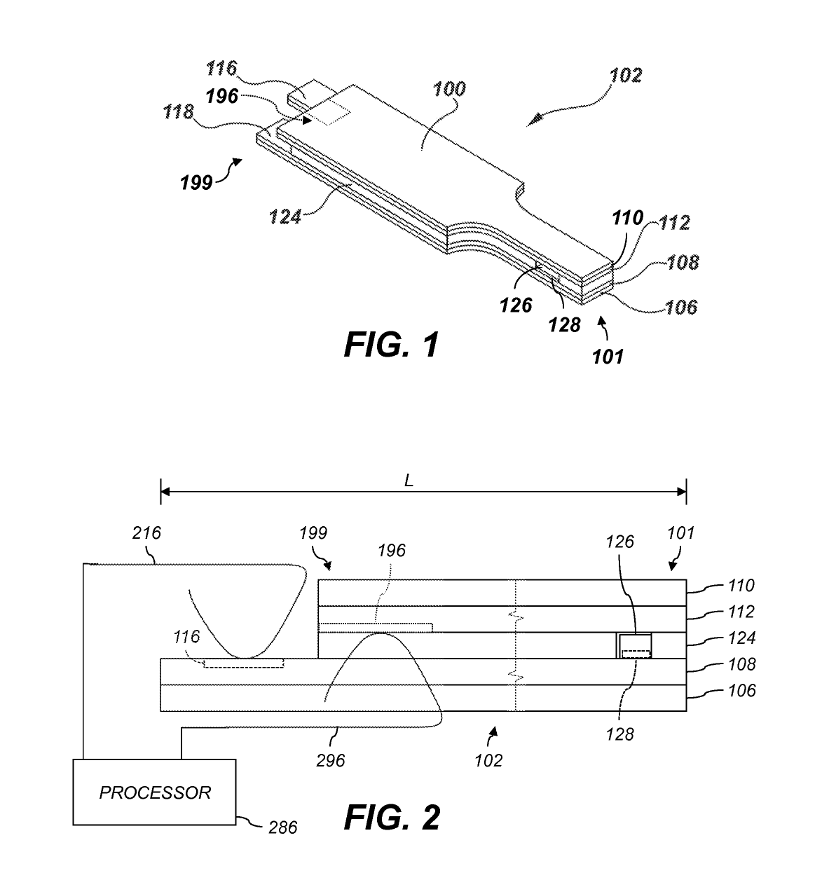

Initially and with reference to FIGS. 1 and 2, there is shown an exemplary integral analytical test strip 102 with a length L (FIG. 2), and related components. The test strip 102 includes an elongate test strip body 100 extending from a proximal end or portion 101 to a distal end or portion 199. The proximal portion 101 of the test strip body 100 includes a sample cell 126 having multiple electrodes and a reagent (e.g., in a reagent layer 128), while the distal portion 199 of the test strip body 100 includes various features for electrically communicating with a test meter. In use, physiological fluid or a control solution can be delivered to the sample cell 126 for electrochemical analysis.

More specifically, the test strip 102 is defined by a first electrode layer 108 and a second electrode layer 112, with a spacer layer 124 being positioned therebetween. The first electrode layer 108 provides a first electrode and a first conductor for electrically connecting the first electrode to an electrical contact 116. Similarly, the second electrode layer 112 provides a second electrode and a second conductor for electrically connecting the second electrode with an electrical contact 196. The insulating layers 106, 110 can support the electrode layers 108, 112 respectively. The insulating layers 106, 110, or the spacer layer 124, can be opaque or transparent, and can be formed from, e.g., plastics (such as PET, PETG, polyimide, polycarbonate, polystyrene), ceramic, glass, silicon, or adhesives. The electrodes, conductors, and electrical contacts 116, 196 can include discrete areas of conductive material, or can be defined areas on a sheet of conductive material.

In the example shown, the electrode layers 108, 112 are conductive sheets having such areas defined. The electrode layers 108, 112 each can be formed from conductive material, such as gold, palladium, carbon, silver, platinum, tin oxide, iridium, indium, and combinations thereof (e.g., indium tin oxide). Carbon in the form of graphene may also be used. The conductive material can be deposited onto the insulating layers 106, 110 by various processes, such as sputtering, electroless plating, thermal evaporation and screen printing. In an exemplary embodiment, the reagent-free electrode, e.g., the electrode layer 112, is a sputtered gold electrode and the electrode layer 108 supporting the reagent layer 128 is a sputtered palladium electrode. As discussed herein and in use, one of the electrode layers can function as a working electrode and the remaining electrode layer can function as a counter or reference electrode.

Each of the electrode layers 108, 112 can include adjacent, electrically-contacting areas of different conductive materials. For example, the electrode layer 108 can include a silver conductor that electrically connects the sputtered palladium electrode to the electrical contacts 116, 118. The electrode layer 112 can include a silver conductor that electrically connects the sputtered gold electrode to the electrical contacts 196.

The sample cell is defined by the first electrode layer 108, the second electrode layer 112, and the spacer layer 124. Specifically, the first electrode layer 108 and the second electrode layer 112 respectively define the bottom and top of the sample cell 126. A cutout area of the spacer layer 124 defines side walls of the sample cell 126, here, the proximal and distal sidewalls. A plurality of ports provide sample inlet(s) or vent(s). For example, one of the ports provides a fluid-sample ingress and the remaining port acts as a vent.

A first electrical contact 116 is provided in the distal portion 199 of the test strip body 100 and is electrically connected to, or is part of, the first electrode layer 108. This contact is used to establish electrical connection to a test meter. A second electrical contact 196 is also provided at the distal portion 199 and can be accessed by the test meter through a U-shaped notch. The second electrical contact 196 is electrically connected to, or is part of, the second electrode layer 112. In the example shown, the first electrode layer 108 also provides a third electrical contact 118 electrically connected to the first electrical contact 116. A test meter can detect the test strip 102 by sensing electrical connection between two contact pins arranged to respectively contact the first electrical contact 116 and the third electrical contact 118. The electrical contacts 116, 118, 196 can be contact pads or have other forms.

The reagent layer 128 is shown with dashed lines in FIG. 2 for clarity. The reagent layer 128 is located wholly or partly in the sample cell 126. The reagent layer 128 can include a mediator and an enzyme, and can be deposited onto or affixed to the first electrode layer 108. Suitable mediators include ferricyanide, ferrocene, ferrocene derivatives, osmium pipyridyl complexes, and quinone derivatives. Suitable enzymes include glucose oxidase, glucose dehydrogenase (GDH) based on pyrroloquinoline quinone (PQQ) co-factor, GDH based on nicotinamide adenine dinucleotide (NAD) co-factor, and FAD-based GDH [E.C.1.1.99.10]. Exemplary reagents and formation processes are described in U.S. Pat. Nos. 7,291,256, 6,749,887, 6,869,441, 6,676,995, and 6,830,934, each of which is incorporated by reference.

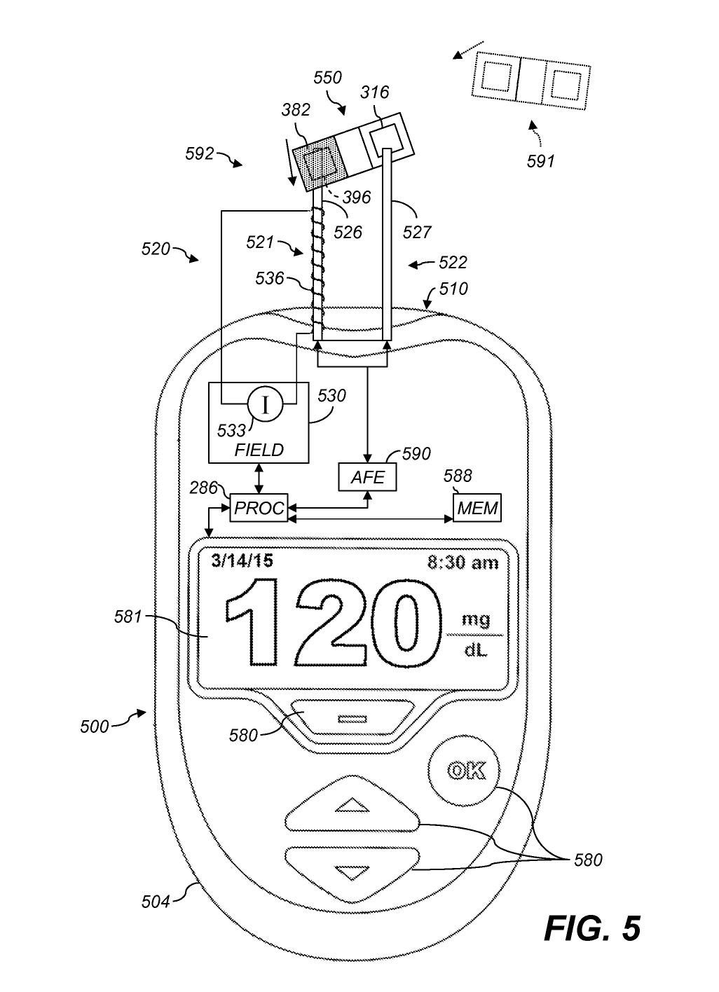

In use, the test strip 102 is configured to interface with a test meter such as shown as 500 in FIG. 5. As depicted for purposes of connection only, the test meter includes a first terminal 216 and a second terminal 296 that are placed in electrical connection with the first electrical contact 116 and the second electrical contact 196, respectively, of the test strip 102. In the example shown, the first terminal 216 and the second terminal 296 are spring contacts arranged so that the test strip 102 can be slid in the direction marked “insertion” to electrically connect the first and second electrical contacts 116, 196 with the first and second terminals 216, 296. The first and second terminals 216, 296 can also include pogo pins, solder bumps, pin or other receptacles, jacks, or other devices for selectively and removably making electrical connections. The test meter is equipped with a processor 286 and optionally other components, e.g., volatile and nonvolatile memory. The test meter is later discussed in greater detail with reference to FIGS. 4 and 5.

Still referring to FIG. 2, the test meter can further include a third terminal (not shown) or other electrical contact arranged to electrically connect to the third electrical contact 118. This permits the test meter to measure the resistance or electrical continuity between the first terminal 216 and the third terminal to detect the test strip 102; continuity is present when the test strip 102 is properly inserted into the test meter.

Once a determination is made that the test strip is electrically connected to the test meter, the test meter can apply a test potential or current, e.g., a constant current, between the first and second electrical contacts. In some examples, once the test meter recognizes the presence of the test strip, the processor 286 is configured to “wake up” and initiate a fluid detection mode.

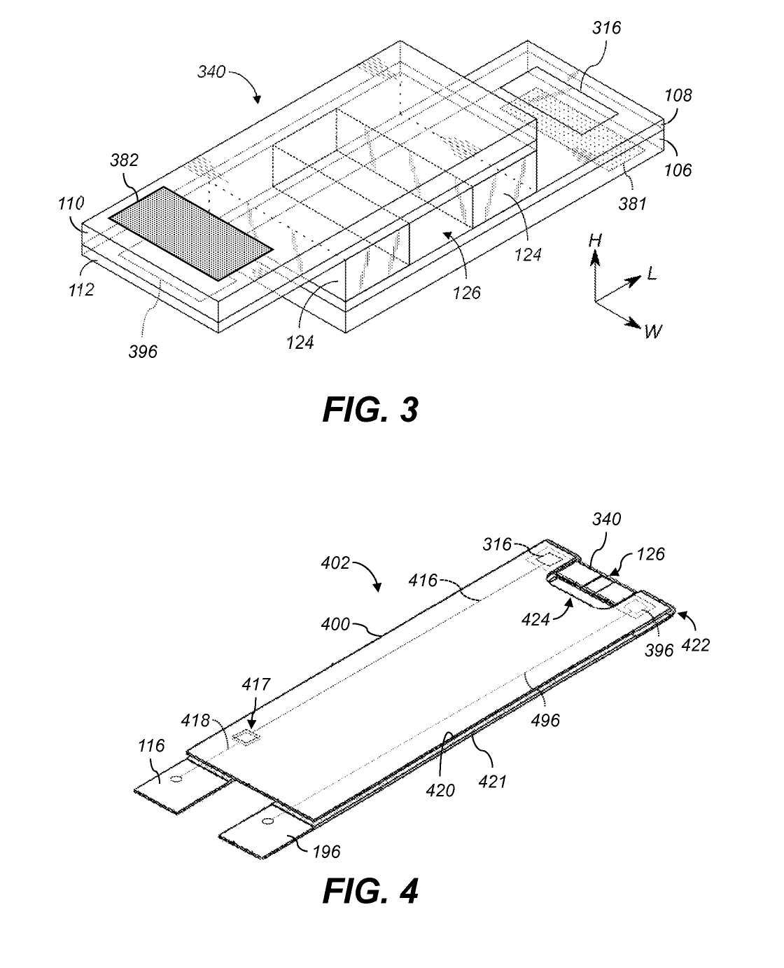

FIG. 3 shows an exemplary electrochemical module (ECM) 340, which is an extremely compact analytical test strip that can be provided either with or without a carrier. Unlike the test strip 102 of FIGS. 1 and 2, the ECM 340 has a first electrode layer 108 and a second electrode layer 112 that are separated by a spacer layer 124 and that protrude at opposite ends of the ECM 340. A first electrical contact 316 is defined on or disposed over the first electrode layer 108 and a second electrical contact 396 is defined on or disposed over the second electrode layer 112. The specific design of the ECM can be defined by a plurality of different configurations, including having other electrode configurations, such as co-planar electrodes. According to this embodiment, magnetic (e.g., ferrous) materials 381, 382 are disposed over the insulating layers 106, 110 respectively, as discussed below with reference to FIG. 5. For clarity, the reagent layer 128, FIG. 1, is not shown. This layer can be arranged in the sample cell 126 as shown in FIGS. 1 and 2.

In one version, the width W of the ECM 340 can be in the range of about 3 mm to about 48 mm, and more preferably about 6 mm to about 10 mm. The length L of the ECM 340 can be in the range of about 0.5 mm to about 20 mm and more preferably about 1 mm to about 4 mm. The distance between the top electrode and the bottom electrode in the height dimension H, as well as the dimensions of the spacer layer 124, can also vary depending on the desired volume of the reaction chamber. In an exemplary embodiment, the sample cell 126 has a small volume. For example, the volume can range from about 0.1 microliters to about 5 microliters, preferably about 0.2 microliters to about 3 microliters, and more preferably about 0.2 microliters to about 0.4 microliters.

Referring to FIG. 4, an exemplary modular analytical test strip 402 is shown. According to the illustrated embodiment, a carrier 400 is provided that retains an electrochemical module (ECM) 340. The carrier 400 can have various configurations, but is typically in the form of one or more rigid or semi-rigid substrates having sufficient structural integrity to support the electrochemical module 340 and to allow handling and connection to a test meter. The carrier 400 is made from a non-conductive and chemically inert material. In this example, the carrier 400 includes a fold line 422 and two rigid portions 420, 421 bendably connected at the fold line 422. The carrier 400 includes at least one hole or opening extending therethrough for providing access to the supported electrochemical module 340. In the illustrated embodiment, the carrier 400 has a single opening 424 located symmetrically across the fold line 422.

The carrier 400 also includes one or more electrical conductors configured to facilitate communication between electrodes on the ECM 340 and a test meter. The electrical conductors can be disposed on all or portions of the carrier 400. In an example, each of the rigid portions 420, 421 of the carrier 400 includes an electrically conducting layer disposed thereon. Each layer can be a conductor, or one or more of the layer(s) can include one or more electrical isolation lines formed (e.g., by laser etching) in the electrically conducting layer(s) to separate each etched layer into multiple mutually-isolated conductors. In the example shown, the first electrical contact 316 of the ECM 340 is electrically connected to an electrical conductor 416 of the carrier 400. Since the illustrated carrier 400 has two rigid portions 420, 421, the electrical conductor 416 is disposed over the rigid portion 420. A bridge 417 electrically connects the electrical conductor 416 to an electrical conductor 418 on the rigid portion 421. The electrical conductor 418 is connected to the first electrical contact 116 on the rigid portion 421. Similarly, the second electrical contact 396 of the ECM 340 is electrically connected to the second electrical contact 196 of the rigid portion 421 via an electrical conductor 496 on the rigid portion 421. In this way, the carrier 400 and the ECM 340 combine to operate as an analytical test strip.

Other arrangements of carriers and ECMs can be used, as can other arrangements of test strips as the foregoing description is intended to be exemplary. For example, the integral analytical test strip 102 of FIGS. 1 and 2 can be reduced in length L to a smaller size. Throughout the remainder of this disclosure, the terms “test strip” and “analytical test strip” refer to integral test strips, in which the sample cell 126 and the electrical contacts 116, 196, all FIG. 1, are constructed in such a way that they cannot be readily separated without damaging the integral test strip; to modular test strips, in which the sample cell 126 and the electrical contacts 116, 196, all FIG. 3, can be readily separated; and to electrochemical modules alone. Magnetic material can be applied to any of these test strips to permit such test strips to operate with test meters described below.

A plurality of test strips, e.g., electrochemical modules, can be stored in a stacked vertical configuration within a container such as a vial, such as described by U.S. Pat. No. 8,016,154 or U.S. Pat. No. 7,712,610, the entire contents of which are herein incorporated by reference. The vial can includes a lid that is releasably and hingeably secured to the upper end of the vial body. Access to the vial can be made by opening the upper lid. The vial can also include an actuator that will present one test strip at a time. The actuator can be, e.g., a motor that drives the topmost test strip in a vertical stack of test strips in the vial out through a slot provided in the side of the vial.

FIG. 5 is a perspective view and block diagram of a test meter 500 and a test strip 550, e.g., an analytical test strip, according to various embodiments. The test meter 500 includes a meter housing 504 that is appropriately sized for retaining a plurality of components including a processor 286 and an analog front end 590 that is configured to apply electrical signals (e.g., voltages) to the test strip 550 and measure electrical signals (e.g., currents) from the test strip 550 in response to the applied signals. The processor 286 can include a controller, such as a microprocessor; a field-programmable gate array (FPGA) such as an ALTERA CYCLONE FPGA; a digital signal processor (DSP) such as a Texas Instruments TMS320C6747 DSP; one or more Application Specific Integrated Circuits (ASICs); or other processing device(s) adapted to carry out algorithm(s) described herein. The processor 286 can be bi-directionally connected by I/O ports to a memory 588 (e.g., including a RAM, ROM, Flash, hard disk drive, or other volatile or nonvolatile storage) and a to plurality of buttons 580 disposed on the exterior of the meter housing 504 that define a user interface along with a display 581 connected to the processor 286. The processor 286 can be configured to, e.g., measure glucose level in blood that has been applied to the test strip 550. The processor 286 can apply a voltage signal to the test strip 550 via the terminals 521, 522, discussed below, and measure a resulting current through the terminals 521, 522, then analyze the current signal to determine the analyte. An example is given in U.S. Pat. No. 8,486,245, incorporated herein by reference in its entirety.

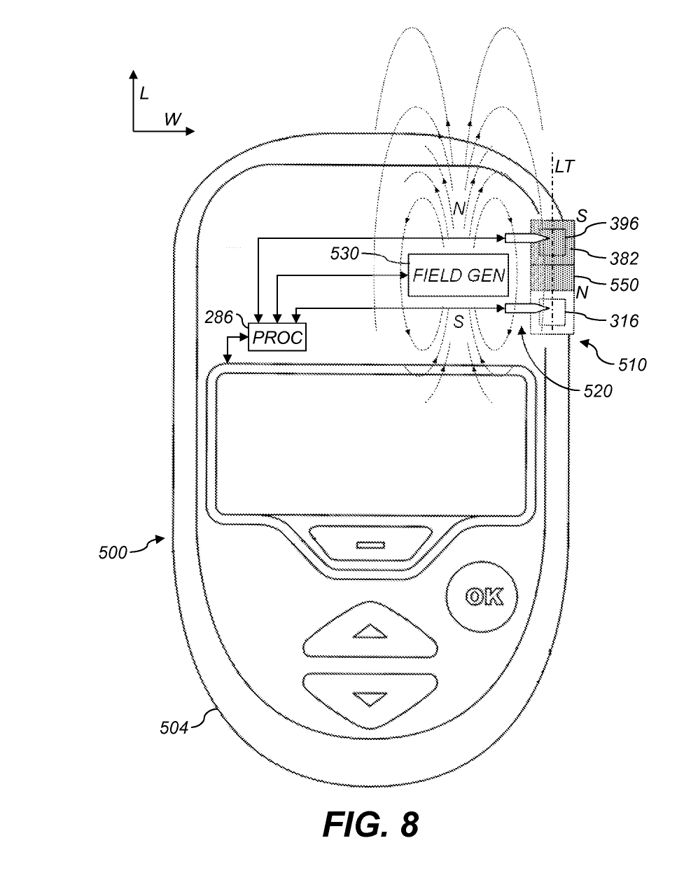

A strip port connector 520 (“SPC”) is provided in or on the meter housing 504. The meter housing 504 has an aperture 510, e.g., a slotted cavity or port, arranged to permit the SPC 520 to receive the test strip 550. The SPC 520 includes a plurality of terminals 521, 522, e.g., including conductive metal prongs, that are suitably aligned or spaced to engage the electrodes of the test strip 550 for purposes of testing a fluid sample. The terminals 521, 522 (any number can be used) can protrude from the meter housing 504, or not. For example and as shown, the terminals 521, 522 can extend outwardly from the meter housing in spaced configuration. The terminals 521, 522 can also be disposed within the meter housing 504, as shown in FIG. 8. The SPC 520 is configured to mechanically and electrically engage the inserted test strip 550, and is operatively connected to the processor 286 to convey electrical signals between the processor 286 or analog front end 590 and the inserted test strip 550. In the example shown, the processor 286 connects via the analog front end 590 to the SPC 520.

As discussed above, a portion of the test strip 550 is provided with an impregnated or otherwise disposed material such as iron or another magnetic material that permits magnetic attraction, but that does not interfere with the testing of the analytical test strip 550 for determining analyte concentration of a fluid sample applied to the strip. As used herein, the term “magnetic” includes ferromagnetic, ferrimagnetic, and paramagnetic materials, and any materials that are attracted by an external magnetic field. Preferably, the magnetic material can be disposed onto the substrate of the test strip as a tape, or the material can alternatively be created using the sputtering or similar process used for manufacturing the electrodes of the analytical test strip. The magnetic material can also be incorporated in an ink that can be printed onto the test strip 550.

A field generator 530 is operatively connected to the strip port connector 520 and the processor 286. The field generator 530 is configured to provide, continuously or selectively, a magnetic field that will draw the magnetic material towards at least one of the terminals, e.g., the terminal 521. A technical effect of the field generator 530 is therefore that, when the field generator 530 is active at the command of the processor 286, and the test strip 550 is close enough to the test meter 500 for the provided magnetic field to overcome frictional and other forces, the test strip 550 will move toward the terminal 521. The field generator 530 can provide the magnetic field, e.g., by energizing an electromagnet.

The field generator 530 can alternatively include one or more magnetic shunts and provide the field by moving a magnet (e.g., a permanent magnet or electromagnet) to direct the field either through the shunts (field not provided) or not (field provided). This alternative is similar to magnetic bases used in optical-bench work and metalworking. Such bases do not provide an external magnetic field when a permanent magnet is oriented so that the N and S poles are aligned in a gap between two spaced-apart iron blocks. When the permanent magnet is rotated so that the N pole is adjacent one block and the S pole is adjacent to the other block, the blocks take on the magnetization of the permanent magnet. The result is that a magnetic field is provided between the two blocks.

Still referring to FIG. 5, in the example shown, the magnetic material 382 is applied to one portion of the test strip 550. The magnetic material 382 can also be applied to two or more spaced-apart portions. The magnetic material 382 can also be applied uniformly across the test strip 550. In any of these configurations, the magnetic material 381 can be arranged in, on, or over the test strip 550 in such a way that the test strip 550 as a whole will align in a selected orientation when placed in the magnetic field, or will have a net magnetization with respect to that field. For example, each piece or region of magnetic material can be composed or oriented to present a specific pole in a selected direction to align or mate with an opposing pole on the SPC. This permits positioning the test strip 550 in a desired orientation with respect to the terminals 521, 522, as is later discussed in greater detail with reference to FIG. 8. In the example shown, the magnetic field is provided with respect to the terminal 521. Magnetic fields can also be provided with respect to the terminal 522 or any number of terminals.

In various embodiments, at least one of the terminals, e.g., the terminal 521, includes an elongated electrical contact 526. The electrical contact 526 can be, e.g., a pin or pogo pin. The field generator 530 includes a conductor 536 coiled around the elongated electrical contact 526 and electrically isolated therefrom. For example, the conductor 536 can be an insulated wire. The field generator 530 also includes a current source 533 responsive to the processor 286 to drive electrical current (direct or alternating, constant or variable) through the conductor 536. The conductor 536 is an electromagnet when the electric current passes through it. The intensity of the magnetic field produced can be selected by controlling the amount of current provided by the current source 533. In the example shown, the magnetic field is provided with respect to the terminal 521. Magnetic fields can also be provided with respect to the terminal 522 or any number of terminals.

The test strip 550 is shown in the process of being moved by the magnetic field from the conductor 536. The test strip 550 in this example is an ECM similar to ECM 340, FIG. 3. First and second electrical contacts 316, 396 are as shown in FIG. 3. Magnetic material 382 is attracted the conductor 536 by the provided magnetic field; the test strip 550 has been magnetically moved from a position 591 to a position 592 in this example. As a result, the electrical contact 526 has been brought into electrical connection with the electrical contact 396 of the test strip 550, and an elongated electrical contact 527 of the terminal 522 has been brought into electrical connection with the electrical contact 316 of the test strip 550.

In one version, the processor 286 is configured to provide a base voltage when a test strip is not detected by the test meter. The meter is further configured to detect the presence of a test strip through measurement of a voltage or current, so that the processor 286 can further detect that the test strip is fully engaged with the strip port connector (e.g., by a continuity measurement), and when a sample is present (e.g., by a capacitance measurement), based on the detection of different voltages and currents. The processor 286 can also be configured to detect when a test strip 550 is connected only to one of the terminals and not the other, e.g., by time-domain reflectometry (TDR), or by detecting a capacitance transient.

The initial detection of the test strip 550 coming into electrical contact, e.g., with the terminal 521, can activate the test meter from an inactive or “sleep” mode. This detection indicates that the test strip is partially engaged with the test meter. The test meter can be configured to increase the intensity of the magnetic field if a test strip is initially detected, but in which the test strip is not fully aligned with the extending contacts. This increase in field intensity can be generated automatically, according to at least one version. Alternatively, the test meter can include a manual control element to adjust field strength, such as a switch, soft key button or other user actuated feature, implemented either mechanically or through software/firmware in the processor 286. For example, the processor 286 can receive a wake-up command, e.g., via a user press of one of the buttons 580. The processor 286 can then command the field generator 530 to provide a magnetic field of a selected intensity. If the processor 286 does not detect connection of a test strip to both of the terminals 521, 522 within a certain time, the processor 286 can direct the field generator 530 to increase the strength of the magnetic field to draw the test strip 550 more strongly towards the strip port connector 520. That is, the test meter 500 can be configured to detect the presence of a test strip 550 based on electrical contact with at least one terminal 521 of the strip port connector 520. The test meter 500 can be further configured to increase the intensity of the magnetic field based upon detection of a test strip 550. The processor 286 can be configured to automatically increase the intensity of the magnetic field upon detection of the test strip 550.

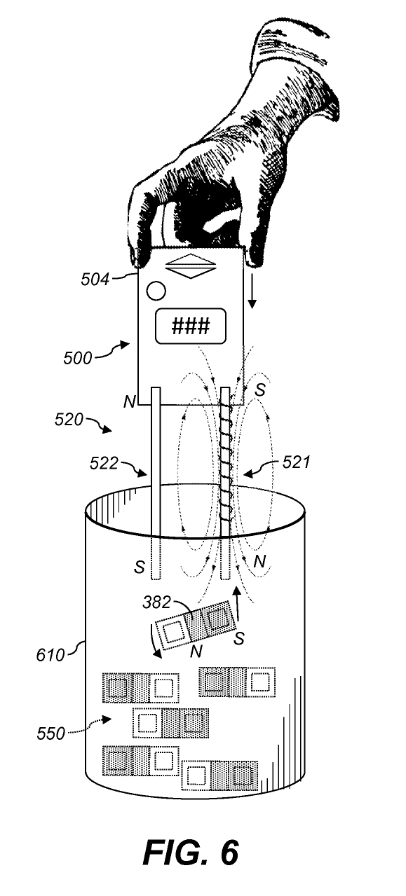

FIG. 6 shows an example of use of the test meter 500. In this example, the test meter 500 includes the strip port connector 520 with the terminals 521, 522 that extend outwardly from the meter housing 504. The terminals 521, 522 are configured for fitting into or otherwise engaging a container 610 of test strips 550. In the example shown, the container 610 is a vial. The container 610 can also be, e.g., a box, carton, reeled or cut tape or other bandolier-type container, or canister. In this example, the strip port connector 520 has been partially inserted into the container 610, e.g., by a user. The size of the container can be easily varied provided the terminals can gain access to the contents thereof.

The magnetic field (shown dashed for clarity) provided via the terminal 521 has a north pole N and a south pole S. That field causes the magnetic material 382 on one of the test strips 550 to be attracted towards the terminal 521. In the example shown, the magnetic material 382 is a permanent magnet with the indicated N and S poles. The magnetic material 382 can also be, e.g., paramagnetic. In this way, a test strip 550 is drawn towards the strip port connector 520. A second magnetic field can be provided, e.g., with respect to the terminal 522, having an orientation different from that of the magnetic field in operative arrangement with the terminal 521. For example, a magnetic field provided by a coil (not shown) around the terminal 522 can have the south pole S closer to the container 610, and the north pole N closer to the meter housing 504, as indicated. This can provide magnetic-field lines that extend between the terminals 521, 522 at their distal ends (away from the meter housing 504), which can encourage the test strip 550 to align perpendicular to the terminals 521, 522.

When the strip port connector 520 is removed from the container 610, e.g., by a user, the test strip 550 is brought with it and a fluid sample can be applied. The test strip 550 can be retained in position with respect to the strip port connector 520 by magnetic forces or by mechanical retention devices such as latches, clips, adhesives, or fasteners.

Still referring to FIG. 6, an analytical testing system can include the test meter 500 with the meter housing 504 retaining the processor 286 and the analog front end 590, both FIG. 5. The system includes a plurality of test strips 550. Each of the test strips 550 (which can be an integral or modular strip, as discussed above) is defined by a substrate (e.g., the insulating layers 106 and 110, FIG. 2) having at least two contact pads (e.g., the electrical contacts 116, 196, FIG. 2), at least two electrodes (e.g., the electrode layers 108, 112, FIG. 2), and a sample cell 126, FIG. 2. A magnetic material 382, FIG. 3, is disposed on the substrate.

The test meter 500 includes at least two terminals 521, 522 disposed from the strip port connector 520. The terminals 521, 522 are configured for engaging the contact pads of a test strip. The terminals 521, 522 can extend outwardly from the meter housing 504 of the test meter 500, or can be disposed within the meter housing 504. A magnetic field is generated in operative arrangement with at least one of the terminals 521, 522, e.g., as discussed above. This operative arrangement can be any field orientation, shape, or strength that will draw the test strip 550 towards the at least one of the terminals 521, 522 so that the electrical contacts 116, 196, FIG. 2, can make electrical contact with the terminals 521, 522 and signals can be passed through the sample cell 126, FIG. 2, by the analog front end 590, FIG. 5.

In various embodiments, the test meter 500 is configured to determine the presence of a test strip 550 based on a detected signal from one of the contacts, e.g., a TDR signal. The test meter 500 is configured to increase the intensity of the magnetic field to attract and align the test strip 550 in a preferred orientation, e.g., across the terminals 521, 522. For example, the processor 286 can be configured to automatically increase the intensity of the magnetic field upon detection of the presence of the test strip 550.

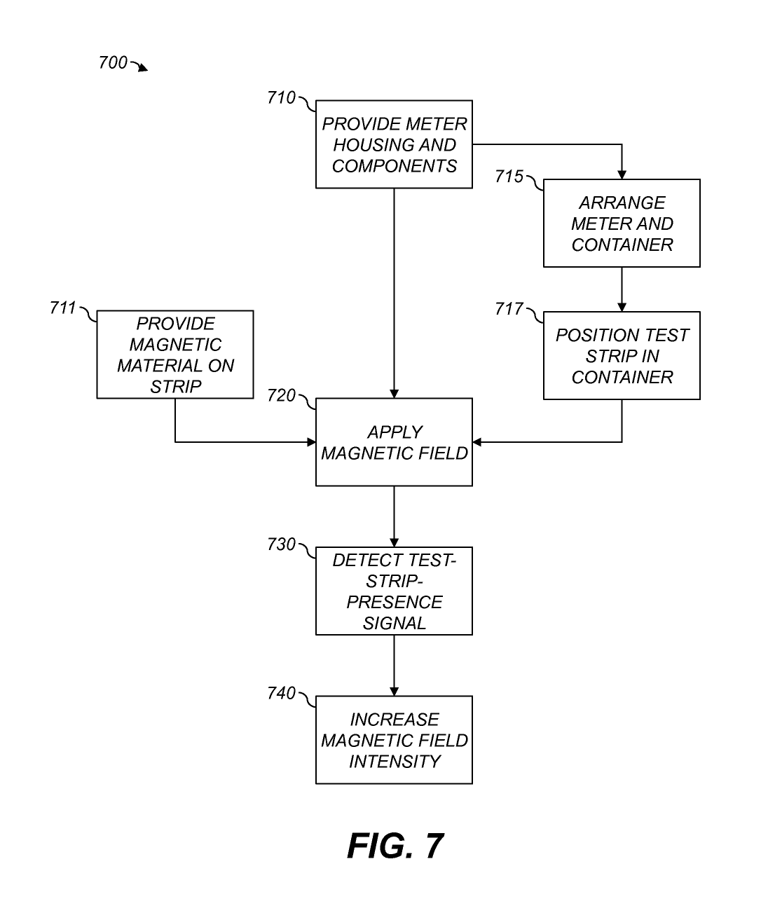

FIG. 7 is a flow diagram depicting stages in an exemplary method for enabling a compact analytical test strip to be accurately inserted into a test meter. Reference is made to various components described above for exemplary purposes. Methods described herein are not limited to being performed only by the identified components.

A method 700, at step 710, includes providing a meter housing 504, FIG. 5. The meter housing 504 has a defined aperture 510, FIG. 5, sized to receive an analytical test strip. A field generator 530, FIG. 5, is connected to terminals of a strip port connector 520, FIG. 5. The test meter 500, FIG. 5, can, e.g., include at least two of the terminals 521, 522, FIG. 5, in spaced relation and extending outwardly from the aperture 510. The test meter 500 can also or alternatively include at least two of the terminals in spaced relation and disposed within the aperture 510 of the meter housing 504.

In various embodiments, at step 715, the strip port connector 520 of the test meter 500 is operatively arranged with respect to a container 610, FIG. 6, holding one or more test strip(s) 550. In this way, the terminals 521, 522 are operatively arranged to receive the test strip 550 from the container 610 of test strips 550. At step 717, the container 610 positions (e.g., presents or dispenses) exactly one test strip 550 so that, when the magnetic field is applied, only that one test strip 550 will be free to move towards the strip port connector 520. Step 717 can include, e.g., the container 610 operating an actuator to move one of the test strips 550 partially off the top of a stack of test strips 550 in the container 610, similar to the operation of a PEZ candy dispenser.

At step 711, in various embodiments, magnetic material is provided on at least a selected portion of the analytical test strip before applying the magnetic field in step 720.

At step 720, a magnetic field is applied in operative arrangement with at least one terminal 521, 522 of the strip port connector 520 to attract at least one of the electrical contacts 116, 196, FIG. 1, of a test strip 550, FIG. 5.

At step 730, in various embodiments, a signal indicative of the presence of a test strip 550 is detected. At step 740, the intensity of the magnetic field is increased based on the detection signal, thereby drawing the analytical test strip into a preferred orientation for conduction of an analyte measurement in which sample is applied and tested, in a manner previously discussed.

Once apprised of the present disclosure, one skilled in the art will recognize that methods according to embodiments of the present invention, including method 700, can be readily modified to incorporate any of the techniques, benefits and characteristics of hand-held test meters according to embodiments of the present invention and described herein. For example, if desired, an analyte in the introduced bodily fluid sample can be determined using the test strip 550 and test meter 500.

FIG. 8 is a perspective view of another embodiment of a test meter 500 and a test strip 550. According to this embodiment, the field generator 530 provides a magnetic field originating from within the meter housing 504. This field (with N and S poles as indicated) draws the test strip 550 (with its N and S poles as indicated) wholly or partly through the aperture 510, with the long axis LT of the test strip 550 oriented substantially in a direction L parallel to the long axis of the meter housing 504. Other orientations of magnetic field and test strip 550 can also be used. For example, the field generator 530 can provide a magnetic field with N and S poles aligned along a direction W perpendicular to the direction L. In this example, the magnetic field causes the test strip 550 to move so that the long axis LT is substantially parallel to the direction W.

In the example shown, the strip port connector 520 includes two terminals. The terminals are represented graphically as pins and, for clarity, are not individually labeled. Each of the terminals includes an electrical contact, e.g., a pin, pogo pin, wiper or other edge connector, or conductive ball, spring, or other conductor. The magnetic field draws the test strip 550 into operative engagement with the strip port connector 520. In this arrangement, each of the terminals makes electrical contact with a respective one of the contact pads of the test strip 550, as shown. It is not required that the test strip 550 be entirely within the meter housing 504. In a preferred embodiment, at least a portion of the test strip 550 protrudes from the meter housing 504 through the aperture 510, or is accessible via the aperture 510, so that a fluid sample can be applied to the sample cell 126, FIG. 3.

The magnetic material 382 can be arranged on the test strip 550, or formulated (e.g., by controlling its magnetic domains), so that one of the electrical contacts 316, 396 will always be at the N pole and the other will always be at the S pole. For example, the contact for a Pd electrode can be at the N pole and the contact for an Au electrode can be at the S pole. This permits the processor 286 to know, when both terminals are in contact with the test strip 550, which terminal is connected to which electrode of the test strip 550.

### PARTS LIST FOR FIGS. 1-8

100 test strip body
101 proximal portion
102 test strip
106 insulating layer
108 electrode layer
110 insulating layers
112 electrode layer
116, 118 electrical contacts
124 spacer layer
126 sample cell
128 reagent layer
196 electrical contact
199 distal portion
216 first terminal
286 processor
296 second terminal
316 electrical contact
340 electrochemical module (ECM)
381, 382 magnetic materials
396 electrical contact
400 carrier
402 test strip
416 electrical conductor
417 bridge
418 electrical conductor
420, 421 rigid portions
422 fold line
424 opening
496 electrical conductor
500 test meter
504 meter housing
510 aperture
520 strip port connector (SPC)
521, 522 terminals
526, 527 electrical contacts
530 field generator
533 current source
536 conductor
550 test strip
580 buttons
581 display
588 memory
590 analog front end
591 position
592 position
610 container
700 method
710, 711, 715, 717 steps
720, 730, 740 steps

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. References to “a particular embodiment” and the like refer to features that are present in at least one embodiment of the invention. Separate references to “an embodiment” or “particular embodiments” or the like do not necessarily refer to the same embodiment or embodiments; however, such embodiments are not mutually exclusive, unless so indicated or as are readily apparent to one of skill in the art. The word “or” is used in this disclosure in a non-exclusive sense, unless otherwise explicitly noted. It is intended that the following claims define the scope of the invention and that devices and methods within the scope of these claims and their equivalents be covered thereby.

What is claimed is:

1. A test meter configured to receive an analytical test strip, the analytical test strip comprising at least one planar substrate having a magnetic material applied to at least a portion thereof, the meter comprising:
a meter housing;
a strip port connector including at least two terminals, the strip port connector arranged with respect to a housing aperture to receive the analytical test strip;
a processor disposed within the meter housing and operatively connected to the strip port connector; and
a field generator operatively connected to the strip port connector and the processor, the field generator being configured to provide a magnetic field that will draw the magnetic material towards at least one of the terminals.

2. The test meter as recited in claim 1, wherein the processor is configured to receive a signal based on electrical contact of the analytical test strip with at least one terminal of the strip port connector.

3. The test meter as recited in claim 2, wherein the processor is programmed to increase the intensity of the provided magnetic field to align the analytical test strip in a predetermined orientation relative to the test meter.

4. The test meter as recited in claim 3, wherein the processor is programmed to automatically increase the intensity of the magnetic field in response to the signal.

5. The test meter as recited in claim 1, wherein the terminals extend outwardly from the meter housing in a spaced configuration.

6. The test meter as recited in claim 5, wherein the outwardly extending terminals are configured for engaging a container of analytical test strips.

7. The test meter as recited in claim 1, wherein the terminals of the strip port connector are disposed within the meter housing.

8. The test meter as recited in claim 1, wherein the magnetic material is applied to at least two spaced-apart portions of the analytical test strip.

9. The test meter as recited in claim 1, wherein:
the analytical test strip further comprises at least one contact;
the at least one of the terminals includes an elongated electrical contact; and
the field generator includes a conductor coiled around the elongated electrical contact and electrically isolated therefrom, and a current source responsive to the processor to drive electrical current through the conductor, so that a magnetic field is produced that draws the contact of the analytical test strip towards the elongated electrical contact.

10. An analytical testing system comprising:
a test meter comprising a meter housing retaining a processor;
a plurality of analytical test strips, each of the test strips defined by one or more substrate(s), two contact pads disposed over the substrate(s), and a magnetic material disposed on at least one of the substrate(s);
the meter including two electrical terminals disposed from a strip port connector and configured for engaging the contact pads of a test strip, and in which a magnetic field is generated in operative arrangement with at least one of the terminals.

11. The system as recited in claim 10, in which the test meter is configured to determine the presence of a test strip based on a detected signal from one of the terminals and in which the meter is configured to increase the intensity of the magnetic field to attract and align the test strip in a preferred orientation.

12. The system as recited in claim 11, wherein the processor is configured to automatically increase the intensity of the magnetic field upon detection of the presence of the test strip.

13. The system as recited in claim 10, wherein the terminals extend outwardly from the meter housing of the test meter.

14. The system as recited in claim 10, wherein the terminals are disposed within the meter housing of the test meter.

15. A method for enabling an analytical test strip to be accurately inserted into a test meter, the method comprising:
providing the test meter including a meter housing having an aperture and a field generator connected to at least one terminal of a strip port connector; and
applying a magnetic field in operative arrangement with the at least one terminal of the strip port connector using the field generator to attract a portion of an analytical test strip.

16. The method as recited in claim 15, including the step of providing magnetic material on at least a selected portion of the analytical test strip before applying the magnetic field.

17. The method as recited in claim 16, wherein attraction of the analytical test strip causes the analytical test strip to be placed in a predetermined orientation relative to the test meter.

18. The method as recited in claim 15, in which a plurality of analytical test strips are retained in a container, the method further comprising operatively arranging the terminals to receive the test strip from a container of test strips.

19. The method as recited in claim 15, further comprising:
detecting a signal indicative of the presence of an analytical test strip; and
increasing the intensity of the magnetic field based on the detected signal.

20. The method as recited in claim 18, wherein the test meter includes two of the terminals in spaced relation and disposed within the aperture.
